(19) United States
(12) Patent Application Publication
(10) Pub. No.: US 2011/0279056 A1
(43) Pub. Date: Nov. 17, 2011

(54) LIGHT EMITTING DIODE LIGHTING MODULE FOR SURGICAL OR DENTAL HANDPIECE

(75) Inventors: Gabriel Waelti, Sonceboz-Sombeval (CH); Edgar Schoenbaechler, Neuchatel (CH)

(73) Assignee: BIEN-AIR HOLDING SA, Bienne 6 (CH)

(21) Appl. No.: 13/105,525

(22) Filed: May 11, 2011

(30) Foreign Application Priority Data
May 11, 2010 (EP) 10162562.2

(51) Int. Cl. H05B 37/02; A61B 1/06

(52) U.S. Cl. 315/291; 362/119

(57) ABSTRACT

Lighting module (1) with an LED (4) for a handpiece for dental or surgical use, characterized in that said lighting module (1) includes a current source (2) delivering an operating current (I1) to an LED (4), wherein the current source (2) and the LED (4) are encapsulated in the same enclosed volume (V), and the lighting module (1) includes electrical connection means for the connection thereof to an electrical power grid.

This application claims priority from European Patent Application No. 10162562.2 filed May 11, 2010, the entire disclosure of which is incorporated herein by reference.

### FIELD OF THE INVENTION

The present invention concerns a light emitting diode (LED) lighting device intended to be fitted to a handpiece for dental or surgical use. The present invention concerns in particular a lighting device allowing a filament light bulb to be replaced in an existing handpiece by an LED without any need to alter the electric cables of the handpiece or the unit which controls the handpiece and which supplies it with power and fluids.

### BACKGROUND OF THE INVENTION

Most dental or surgical instruments are fitted with a lighting device including a light source for lighting the working area of the instrument, which makes the practitioner's task considerably easier by enabling him to enjoy a clear view of the operating field. Until recently, the light source was formed by a filament bulb, the operation of which is well-proven. However, for some time now, the tendency of manufacturers of handpieces for dental or surgical use is to replace filament bulbs with LEDs. The advantages of LEDs relative to conventional filament bulbs are known: improved efficiency (in other words, for the same lighting intensity, the electric power consumption of a diode is less than that of a bulb), increased lifetime and the production of light which is closer to natural light.

Various solutions have been adopted in order to replace filament bulbs with LEDs. The most trivial of these solutions consists simply in exchanging the filament bulb for an LED. In some cases, this solution simply does not work. Indeed, below a threshold voltage, the diode is not lit. Consequently, the electronic circuit of the handpiece or unit to which the handpiece is connected must be capable of supplying the diode with a supply voltage higher than the threshold voltage of the diode, which is not always the case. Moreover, even when the handpiece or unit is capable of supplying the LED with a higher voltage than the threshold voltage, it is known that the current in a diode increases very rapidly according to the voltage. Consequently, if the intensity of the current in the diode is not monitored, the risk of exceeding the current value acceptable to the diode, and therefore destroying the diode, is significant.

To overcome this latter problem, it has already been proposed to arrange a resistor in series with the LED. This solution has the merit of limiting the current in the diode and thus limiting the risks of destroying the diode by a current that is too intense. However, the operation of this arrangement is dependent upon the voltage and intensity values of the current supplied by the handpiece or by the unit to which the handpiece is connected and is not therefore reproducible. Thus, depending upon the equipment that one practitioner or another has, the operation of the LED is random, which is unacceptable.

Another solution consists in integrating, from the outset, a conversion circuit in the handpiece in order to supply the diode with current. However, this is a solution which cannot be implemented in an existing instrument and which requires the practitioner to purchase a new instrument, which is expensive for the practitioner from the point of view of economy.

### SUMMARY OF THE INVENTION

It is an object of the present invention to overcome the aforementioned drawbacks, in addition to others, by providing an LED lighting device, which guarantees reliable and reproducible operation of the LED, yet can be integrated in an existing handpiece in place of a filament bulb.

The present invention therefore concerns an LED lighting module for a handpiece for dental or surgical use, characterized in that said lighting module includes a current source delivering an operating current to an LED, the current source and the LED being encapsulated in the same enclosed volume, and the lighting module including connecting means for connection to an electric power grid.

Owing to these features, the present invention provides an LED lighting module wherein the elements necessary for the proper operation of the diode and the diode itself are housed in the same enclosed volume. A discrete component is thus obtained, the operation of which is reproducible, regardless of the electrical features of the handpiece in which it is integrated. This discrete component forms a removable, disposable element which can be replaced, a supply device integrated in the consumable element being inseparable of the LED.

According to a complementary feature of the invention, the lighting module forms a unit that can be interchanged part for part with a filament bulb of the type used in existing handpieces.

According to yet another feature of the invention, the lighting module is adapted as regards the shapes, dimensions and the electrical connecting means thereof, so that it can be substituted for a filament bulb used in existing handpieces.

Owing to these other features, the LED lighting module according to the invention can advantageously replace part for part a commercial filament bulb fitted to an existing handpiece. The present invention thus advantageously allows existing handpieces to be brought to a higher standard of quality by replacing a filament bulb with an LED without requiring any mechanical or electrical alteration to such handpieces. The user thus has available a handpiece whose lighting device has a longer lifetime and produces lighting very close to that of natural light, without being required to purchase a new handpiece. The user needs only purchase the lighting module according to the invention and connect it instead of the filament bulb originally fitted to the handpiece. Since the lighting module occupies the same volume as a filament bulb and the electrical connections thereof are arranged at the same place as in the bulb, replacing this bulb with a lighting module according to the invention does not require any mechanical or electrical adaptations.

According to a first variant of the invention, the current source is powered from outside the enclosed volume by a voltage source and includes means for converting the voltage supplied by the external voltage source into operating current for the LED.

The voltage source may be a variable voltage source. In such case, a current source which produces a current that varies according to the input voltage is used. This is necessary in the case where the intensity of the lighting provided by the diode is required to be as high as the applied voltage is large.

If the voltage source used is a variable voltage source, a current control and limiting device is connected to the current source so as to define a ratio of proportionality between the value of the applied voltage and the intensity of the current supplied by the current source which determines the lighting intensity produced by the LED.

According to a second variant of the invention, the current source is powered from outside the enclosed volume by a current source delivering a current of given intensity and includes means for varying the current of given intensity so as to obtain the LED operating current.

According to a complementary feature of the invention, upstream of the current source, the lighting module includes a rectifier element which means that the LED can be arranged regardless of polarity.

According to another feature of the invention, a voltage overload protection element is inserted between the rectifier element and the current source. The purpose of this element is to protect the lighting module according to the invention against any overvoltage which may occur at the input of the lighting module in the event of misuse by the practitioner. Downstream of the current source, an element for protection against absence of load can also be provided. The purpose of this protection element is to protect the lighting module in the event that the diode is absent. Indeed, if the diode is missing or out of service, the voltage may increase to values that would destroy the module.

According to yet another feature of the invention, a first filter is inserted between the rectifier element and the overvoltage protection element for filtering the electromagnetic interference generated by the operation of the handpiece and which interfere with the current source. Downstream of the current source, after the load absence protection element, a second filter may be provided, the purpose of which is to stabilise and smooth the current produced by the current source and to filter the interference inherent to the noise caused by the operation of the current source. Indeed, the current source is typically a chopping current source operating at a frequency of 1 MHz.

In one embodiment of the invention, the LED and the control module associated therewith are inserted in a metal, plastic or ceramic tube which defines a hermetically sealed volume, the tube being closed at the base by an end piece and including at the top end thereof, on the side of the LED, a collimation lens which is bonded or crimped onto the tube.

Another technique of encapsulating the lighting module of the invention consists in taking a container, into which the LED and the electronic control circuit thereof are inserted, then pouring resin into the container so as encapsulate the printed circuit board on which the components necessary for operation of the LED are mounted, with the LED emerging from the container.

Advantageously, the metal tube or the container in which the lighting module according to the invention is inserted occupies a similar volume to that of an ordinary filament bulb, which further simplifies replacement of this type of filament bulb with the LED.

### BRIEF DESCRIPTION OF THE DRAWINGS

Other features and advantages of the present invention will appear more clearly from the following detailed description of one embodiment of the LED lighting device according to the invention, this example being given solely by way of non-limiting illustration with reference to the annexed drawing, in which:

FIG. 1 is a schematic diagram of the lighting module according to the invention imprisoned in an enclosed volume;

FIG. 2 is a similar view to that of FIG. 1 wherein the current source is formed by a voltage source external to the enclosed volume and which powers the LED via a voltage-current converter;

FIG. 3 is a similar view to that of FIG. 2 wherein the external voltage source is a variable voltage source, a current control and limiting device being connected to the current source so as to define a ratio of proportionality between the applied voltage value and the intensity of the current supplied by the current source, which determines the intensity of the lighting produced;

FIG. 4 is a similar view to that of FIG. 3 wherein the lighting module includes a rectifier element between the voltage source and the current source;

FIG. 5 is a similar view to that of FIG. 4 wherein a first overvoltage protection element is inserted between the rectifier element and the current source, and a second load absence protection element is arranged downstream of the current source;

FIG. 6 is a similar view to that of FIG. 5 wherein a first filter for filtering the electromagnetic interference generated by the operation of the handpiece is inserted between the rectifier element and the first overvoltage protection element, a second filter, the purpose of which is to stabilise and smooth the current produced by the current source, is arranged downstream of the current source, after the load absence protection element;

FIG. 7 is a similar view to that of FIG. 1 wherein the current source of the lighting module according to the invention is powered from outside the enclosed volume by an external current source and includes means for varying the current intensity so as to obtain the LED operating current;

FIGS. 8A to 8C are views of a first encapsulation variant of the lighting module according to the invention wherein the lighting module is inserted into a hermetically sealed metal, plastic or ceramic tube;

FIG. 9 is a view of a second encapsulation variant of the lighting module according to the invention wherein the lighting module is inserted into a container which is filled with resin, and

FIG. 10 is a schematic diagram of a commercial filament bulb which illustrates that the lighting module according to the invention has the same shapes and dimensions and the same electrical connection arrangement as this type of bulb.

### DETAILED DESCRIPTION OF THE PREFERRED EMBODIMENTS

The present invention proceeds from the general inventive idea which consists in encapsulating at least one LED and the current source associated therewith in the same enclosed volume. A discrete lighting component is thus obtained, the operation of which is completely independent of the electrical features of the handpiece in which it is integrated. Further, the discrete component can replace a commercial available filament bulb part for part, without any need to alter the existing handpiece either from the mechanical or electrical point of view. It is thus possible to bring existing handpieces to a higher standard without the user incurring the extra cost linked to purchasing a new handpiece.

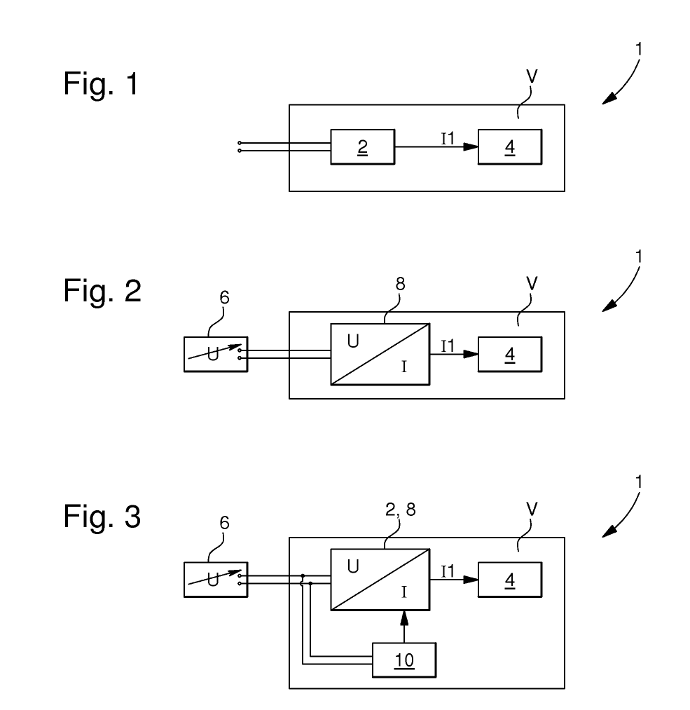

Designated as a whole by the general numerical reference 1, the lighting module according to the present invention includes (see FIG. 1) a current source 2 delivering an operating current I1 to an LED 4. According to the invention, the LED 4 and the current source 2 associated therewith are integrated in an enclosed volume V as will be described in detail below.

Current source 2 is powered from outside the enclosed volume V by a voltage source 6 and includes means 8 for converting the voltage U supplied by the external voltage source into operating current I1 for the LED (see FIG. 2). Of course, the voltage conversion means 8 is integrated in the same enclosed volume V as the lighting module to which it belongs.

The voltage source 6 may be a variable voltage source. In such case, a current source 2 producing a current I1 which varies according to the input voltage U is used. This is necessary if the lighting intensity supplied by diode 4 is required to be as high as the applied voltage U is large. If the voltage source 6 used is a variable voltage source, a device 10 for controlling and limiting the current is connected across current source 2 so as to define a ratio of proportionality between the value of the applied voltage U and the intensity of current I1 supplied by current source 2, which determines the lighting intensity produced by LED 4 (see FIG. 3). Of course, the current control and limiting device 10 is integrated within the same enclosed volume V as current source 2 and LED 4.

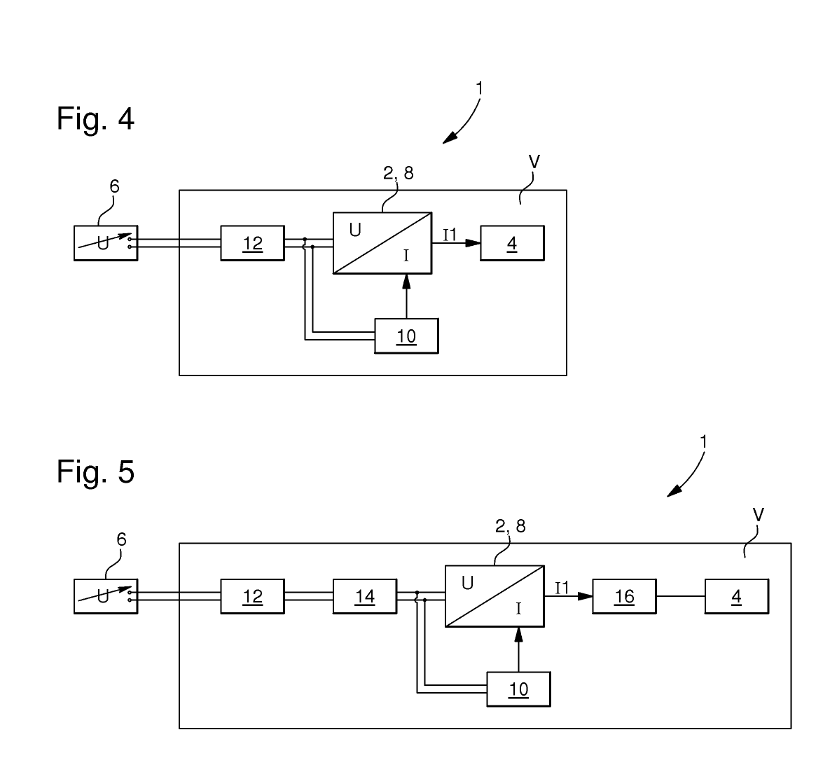

According to a complementary feature of the invention, between voltage source 6 and current source 2, lighting module 1 includes a rectifier element 12, which enables LED 4 to be arranged regardless of polarity (see FIG. 4). Rectifier element 12 includes a set of four diodes arranged in a Gretz bridge arrangement, which acts such that, whatever the polarity of input voltage U, the polarity of this voltage at the output of the Gretz bridge arrangement is always the same. Of course, rectifier element 12 is integrated in the same enclosed volume V as the lighting module 1 to which it belongs.

According to another feature of the invention, a first voltage overload protection element 14 is inserted between rectifier element 12 and current source 2 (see FIG. 5). The purpose of first protection element 14 is to protect lighting module 1 according to the invention against any overvoltage which may occur at the input of the lighting module in the event of misuse by the practitioner. Downstream of current source 2, a second element 16 for protection against absence of load can also be provided. The purpose of this second protection element 16 is to protect lighting module 1 in the event that diode 4 is absent. Indeed, if diode 4 is missing or out of service, the voltage may increase to values that would destroy the lighting module 1. Of course, the first and second protection modules 14 and 16 are integrated in the same enclosed volume V as the lighting module to which they belong.

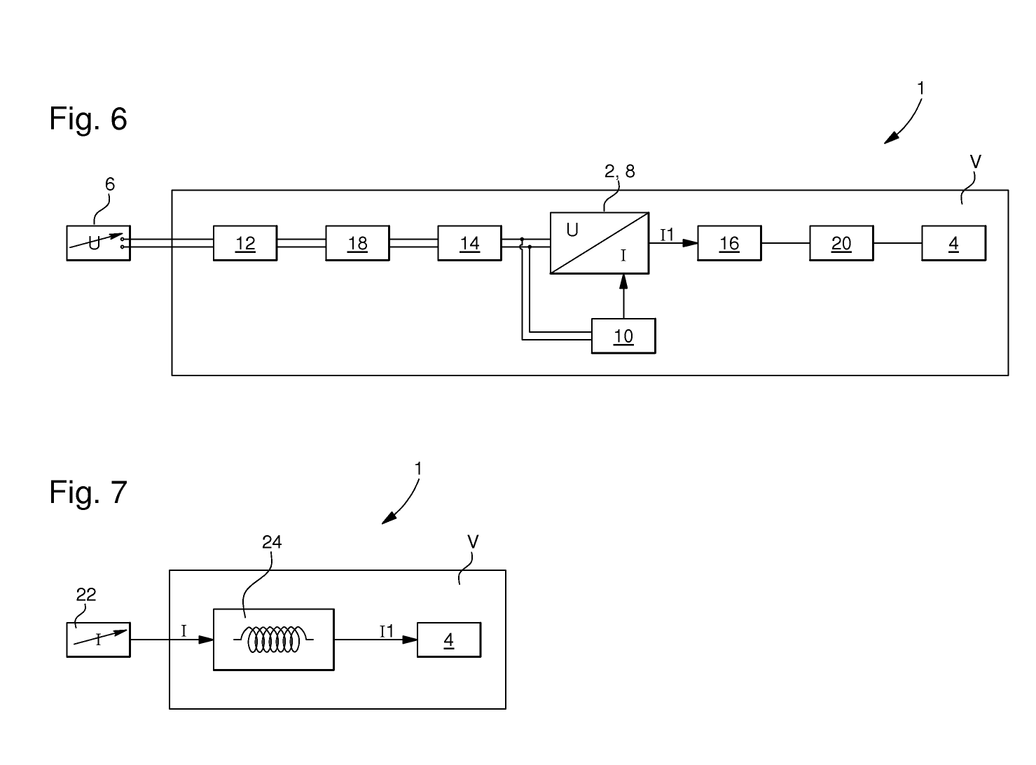

According to yet another feature of the invention, a first filter 18 is inserted between rectifier element 12 and first overvoltage protection element 14 for filtering the electromagnetic interference generated by the operation of the unit to which the handpiece is connected and which interfere with current source 2 (see FIG. 6). Downstream of current source 2, after the load absence protection element 16, a second filter 20 may be provided, the purpose of which is to stabilise and smooth the current produced by current source 2 and to filter the interference inherent to the noise caused by the operation of current source 2. Indeed, current source 2 is typically a chopping current source operating at a frequency of 1 MHz. Of course, the first and second filters 18 and 20 are integrated in the same enclosed volume V as the lighting module to which they belong.

According to a second variant of the invention, current source 2 of lighting module 1 is powered from outside the enclosed volume V by an external current source 22 delivering a current of given intensity I and includes means 24 for varying the current of given intensity I so as to obtain the operating current I1 for LED 4 (see FIG. 7). Of course, the current intensity variation means 24 is integrated in the same enclosed volume V as the lighting module to which it belongs.

We will now consider the techniques for encapsulating lighting module 1 according to the invention as described above in an enclosed volume V.

LED 4 is a discrete component. In one embodiment, it includes an active light emitting element 26 mounted on a first integrated printed circuit board 28 and connected to the conductive paths structured on said printed circuit board by wire bonding 30. Of course, this example embodiment of an LED is given purely for illustrative purposes and other types of LED can be envisaged by those skilled in the art without departing from the scope of the invention as defined by the annexed claims.

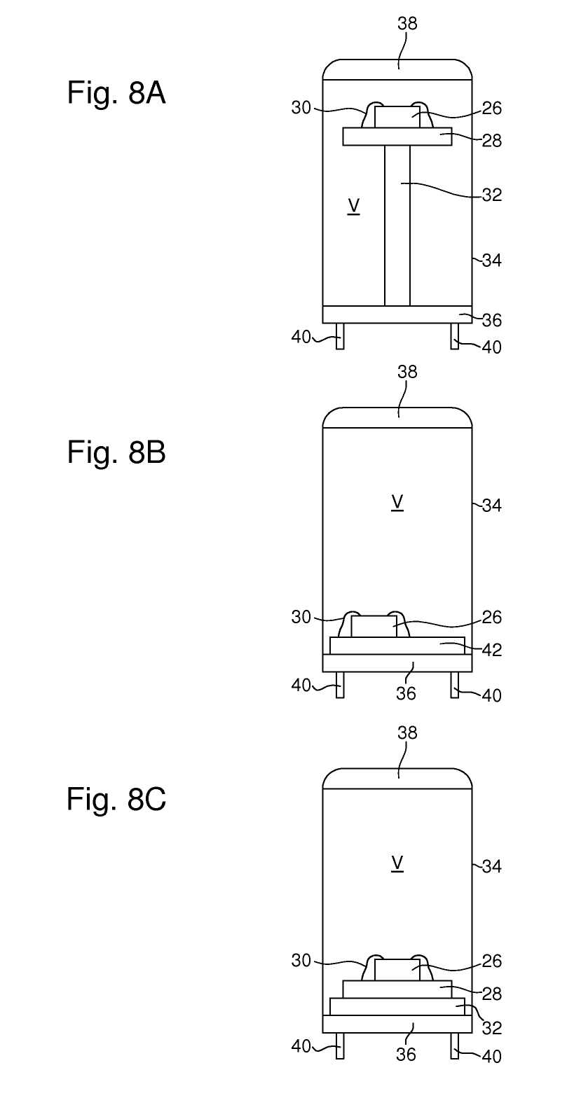

The other components involved in the embodiment of lighting module 1 according to the invention are mounted on a second printed circuit board 32 which is, for example, fixed underneath the first printed circuit board 28 and perpendicular thereto. The unit thus formed can be inserted into a, for example, metal, plastic or ceramic tube 34, which defines a hermetically sealed volume V (see FIG. 8A). Tube 34 is closed at the base thereof by an end piece 36 and includes at the top end thereof, on the side of LED 4, a collimation lens 38 which is bonded or crimped onto tube 34. This unit can be sterilised. The unit is completed by two connection lugs 40 which project from tube 34 at the back thereof and which allow lighting module 1 to be connected to an external electrical power grid.

According to a first variant (FIG. 8B), the LED 4 and the electronic components necessary for the operation of LED 4 are mounted on the same printed circuit board 42.

According to a second variant (FIG. 8C), the first printed circuit board 28, to which LED 4 is secured, is assembled in parallel to the second printed circuit board 32, on which the components necessary for the operation of LED 4 are mounted.

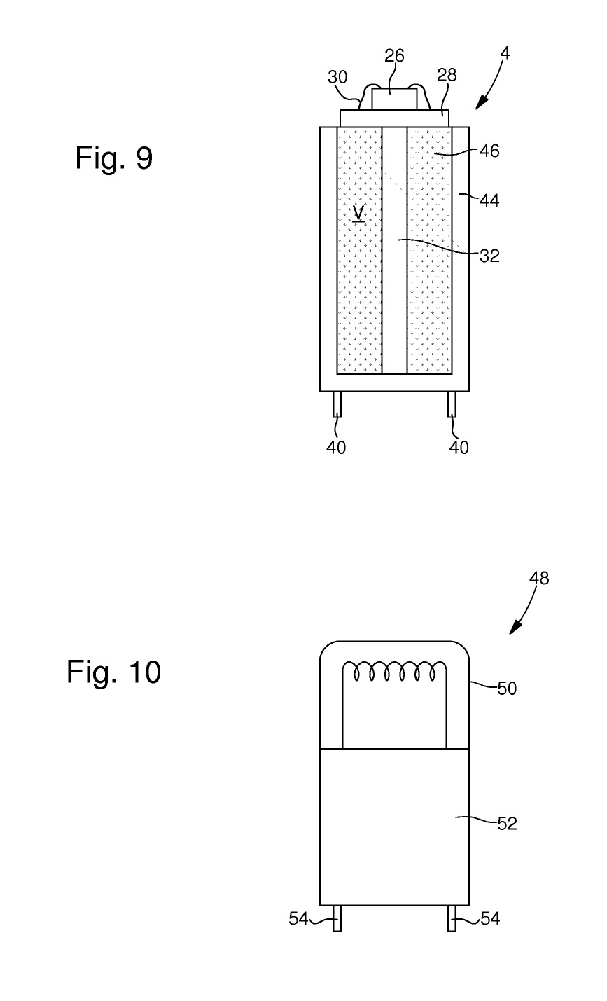

Another technique of encapsulating lighting module 1 according to the invention consists (see FIG. 9) in taking a container 44 into which LED 4 and the electronic circuits thereof are inserted. Resin 46 is then poured into container 44 so as to encapsulate the second printed circuit board 32 on which the components necessary for the operating of LED 4 are mounted, with said LED 4 emerging from container 44. Two connection lugs 40 project from tube 44 at the back thereof and allow lighting module 1 to be connected to an external electrical power grid.

FIG. 10 is a schematic diagram of a commercial filament bulb 48 used in an existing handpiece. A bulb of this type essentially includes a glass bulb 50 extended by a base 52 and two connection lugs 54 for the connection thereof to an external electrical power grid. The identical nature of the shapes and dimensions of filament bulb 48 and lighting module 1 according to the invention will be appreciated, two embodiments of which are illustrated by way of example in FIGS. 8 and 9. Filament bulb 48 and lighting module 1 are rendered completely interchangeable by the fact that connection lugs 40 of lighting module 1 are arranged in the same manner as connection lugs 54 of bulb 48.

The present invention thus provides an LED lighting module forming a disposable, removable element, the inner volume of which houses integrally all of the electronic components necessary for the operation of the LED. It is thus possible to bring an existing handpiece to a higher standard of operation by replacing a commercial filament bulb with an LED component. The user thus has available a handpiece fitted with a lighting device whose lifetime is longer and which produces light very close to natural light, yet without any need to purchase a new handpiece, or even to adapt the handpiece that he already owns from a mechanical or electrical point of view. This remarkable result is achieved owing to the fact that the lighting module according to the invention encloses within the inner volume thereof all of the electronic components necessary for the LED to operate properly, which avoids the need to modify the electrical arrangement of the handpiece and/or the unit to which the handpiece is connected, and owing to the fact that the lighting module according to the invention has identical geometrical and electrical connection features to those of the filament bulb that it replaces, which means that there is no need to alter the mechanical arrangement of the handpiece.

What is claimed is:

1. A lighting module with an LED for a handpiece for dental or surgical use, wherein said lighting module includes a current source delivering an operating current to an LED wherein the current source and the LED are encapsulated in the same enclosed volume, and the lighting module includes electrical connection means for the connection thereof to an electrical power grid.

2. The lighting module according to claim 1, wherein the lighting module forms a unit that can be interchanged part for part with a filament bulb of the type used in existing handpieces.

3. The lighting module according to claim 2, wherein the lighting module is adapted as regards the shapes, dimensions and the electrical connecting means thereof, so that said module can be substituted for a filament bulb used in existing handpieces.

4. The lighting module according to claim 4, wherein the current source is powered from outside the enclosed volume by a voltage source and includes means for converting the voltage supplied by the external voltage source into operating current for the LED.

5. The lighting module according to claim 4, wherein the voltage source is a variable voltage source and wherein the current source produces a current that varies according to the voltage supplied by the variable voltage source.

6. The lighting module according to claim 5, wherein a current control and limiting device is connected across the current source so as to define a ratio of proportionality between the value of the voltage supplied by the variable voltage source and the intensity of the current supplied by the current source, which determines the lighting intensity produced by the LED.

7. The lighting module according to claim 1, wherein, upstream of the current source, the lighting module includes a rectifier element allowing the LED to be arranged regardless of polarity.

8. The lighting module according to claim 4, wherein, upstream of the current source, the lighting module includes a rectifier element allowing the LED to be arranged regardless of polarity.

9. The lighting module according to claim 7, wherein the lighting module includes a first overvoltage protection element inserted between the rectifier element and the current source, said first protection element protecting the lighting module against any overvoltage that may occur at the input of the lighting module.

10. The lighting module according to claim 8, wherein the lighting module includes a first overvoltage protection element inserted between the rectifier element and the current source, said first protection element protecting the lighting module against any overvoltage that may occur at the input of the lighting module.

11. The lighting module according to claim 9, wherein, downstream of the current source, the lighting module includes a second protection element against any overvoltage that occurs in the absence of the LED.

12. The lighting module according to claim 10, wherein, downstream of the current source, the lighting module includes a second protection element against any overvoltage that occurs in the absence of the LED.

13. The lighting module according to claim 11, wherein, inserted between the rectifier element and the first overvoltage protection element, the lighting module includes a first filter for filtering any electromagnetic interference that disturbs the current source.

14. The lighting module according to claim 12, wherein, inserted between the rectifier element and the first overvoltage protection element, the lighting module includes a first filter for filtering any electromagnetic interference that disturbs the current source.

15. The lighting module according to claim 13, wherein, after the second overvoltage protection element, the lighting module includes a second filter, which filters the interference inherent to the noise caused by the operation of the current source.

16. The lighting module according to claim 14, wherein, after the second overvoltage protection element, the lighting module includes a second filter, which filters the interference inherent to the noise caused by the operation of the current source.

17. The lighting module according to claim 7, wherein the current source is powered from outside the enclosed volume by an external current source delivering a current of given intensity and includes means for varying the current of given intensity so as to obtain the operating current for the LED.

18. The lighting device according to claim 1, wherein the lighting module and the LED are inserted into a metal, plastic or ceramic tube which defines a hermetically sealed volume.

19. The lighting device according to claim 17, wherein the lighting module and the LED are inserted into a metal, plastic or ceramic tube which defines a hermetically sealed volume.

20. The lighting module according to claim 18, wherein the tube is closed at the base thereof by an end piece and includes at the top end thereof, on the side of the LED, a collimation lens which is bonded or crimped onto the tube.

21. The lighting module according to claim 19, wherein the tube is closed at the base thereof by an end piece and includes at the top end thereof, on the side of the LED, a collimation lens which is bonded or crimped onto the tube.

22. The lighting module according to claim 1, wherein the lighting module and the LED are placed in a container into which a resin is poured so as to encapsulate the lighting module, with the LED emerging from the container.

23. The lighting module according to claim 18, wherein the lighting module is formed by a printed circuit board on which the components necessary for the operation of the LED are mounted.

24. The lighting module according to claim 20, wherein the lighting module is formed by a printed circuit board on which the components necessary for the operation of the LED are mounted.

25. The lighting module according to claim 22, wherein the lighting module is formed by a printed circuit board on which the components necessary for the operation of the LED are mounted.
